(19) United States
(12) Patent Application Publication
(10) Pub. No.: US 2014/0216130 A1
(43) Pub. Date: Aug. 7, 2014

(54) Simplified method for measuring concentrations of exhaust gas components

(71) Applicant: ENGINE CONTROL AND MONITORING, Los Altos, CA (US)

(72) Inventors: Ronald S. Patrick, Mountain View, CA (US); Robert Lupul, Mountain View, CA (US)

(73) Assignee: Engine Control and Monitoring, Los Altos, CA (US)

(21) Appl. No.: 13/815,146

(22) Filed: Feb. 4, 2013

(51) Int. Cl. G01N 33/00

(52) U.S. Cl. CPC G01N 33/0004
USPC 73/23.31

(57) ABSTRACT

A simplified method for measuring a first property and a second property of an exhaust gas mixture utilizing a first sensor cross-sensitive to a first property and a second property of an exhaust gas mixture, and a second sensor sensitive to the first property, but not to the second property of the exhaust gas mixture. Direct differential measurement between the two sensors quantifies the concentrations of the first property and second property of the exhaust gas mixture.

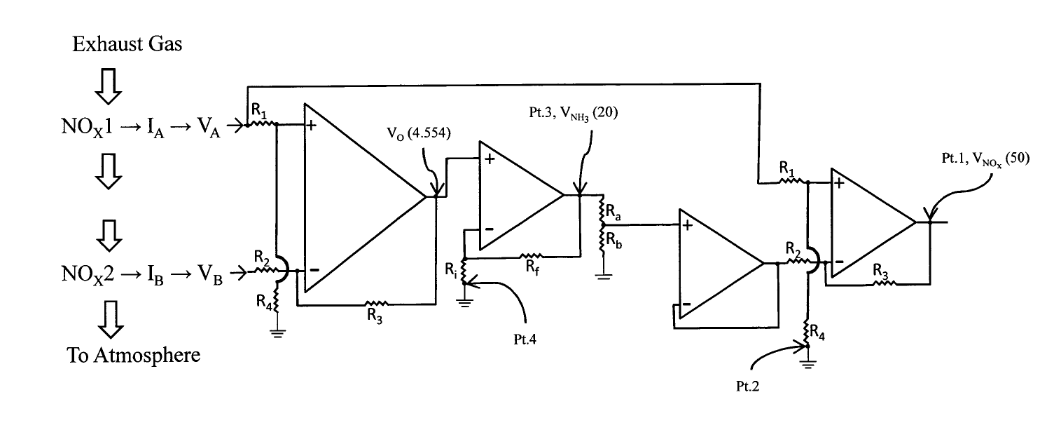

### CROSS-REFERENCE TO RELATED APPLICATIONS

The present application is submitted with reference to, and claims the benefit of, provisional patent application U.S. U.S./797,151 filed on Nov. 30, 2012. The title of the cited provisional application is “Simplified method for measuring concentrations of exhaust gas components”. The text of the first sentence following the title of the specification of the cited provisional patent application is “A simplified method for measuring a first property and a second property of an exhaust gas mixture utilizing a first sensor cross-sensitive to a first property and a second property of an exhaust gas mixture, and a second sensor sensitive to the first property, but not to the second property of the exhaust gas mixture.”.

### STATEMENT REGARDING FEDERALLY SPONSORED RESEARCH

(Not Applicable)

### BACKGROUND OF THE INVENTION

Previous ceramic NOₓ sensors exhibit cross-sensitivities to NH₃. This cross-sensitivity reduces the accuracy of the reported NOₓ concentration from a sensor if NH₃ is also present in the exhaust gas mixture. The disclosed invention covers a simplified method for measuring concentrations of NOₓ and NH₃ in an exhaust gas mixture. Previous inventions have required the use of more than one type of sensor (i.e. NOₓ and NH₃ sensors), or other catalytic components. One example of recent prior art (U.S. Pat. No. 7,810,313) uses at least two sensors in a system, but still requires complex algorithms and a decoupling observer module in order to quantify the relative concentrations of NOₓ and NH₃ in an exhaust gas mixture. The complexity of the above methods is unnecessary and can be reduced significantly in the non-obvious method of the disclosed invention. BRIEF SUMMARY OF THE INVENTION

The disclosed invention covers a simplified method for measuring concentrations NOₓ and NH₃ in an exhaust gas mixture using a NOₓ sensor of traditional design, having a known cross-sensitivity to NH₃, and a second NOₓ sensor not cross-sensitive to NH₃. The design of the second NOₓ sensor uses an integrated NH₃ pre-filter to eliminate the cross-sensitivity to the second property.

### BRIEF DESCRIPTION OF THE DRAWING

The enclosed drawing is a system level diagram of the preferred embodiment of the disclosed invention. Flow of exhaust gas (indicated with bold arrows) in the system as well as the points used for direct differential measurements in an electrical schematic are shown.

### DETAILED DESCRIPTION OF THE INVENTION

A first NOₓ sensor of traditional design having a known NH₃ cross-sensitivity is placed in the exhaust with a second NOₓ sensor that is not cross-sensitive to NH₃. The second NOₓ sensor integrates a NH₃ pre-filter. Direct differential readings between the two sensors are used to determine both NOₓ and NH₃ concentrations simultaneously using the disclosed method: The first NOₓ sensor, having a known cross-sensitivity to NH₃ is placed in an exhaust channel with a second NOₓ sensor that is equipped with a NH₃ pre-filter. A difference in readings from the first NOₓ sensor (NOₓ1) and the second NOₓ sensor (NOₓ2) is determined (NOₓ1−NOₓ2). The resulting value is used to determine the amounts of NOₓ and NH₃ in the exhaust gas mixture. For example: Sensor NOₓ1 has a known, measurable, cross-sensitivity to NH₃ of c₁. Possible NH₃ cross-sensitivity values range from greater than zero to 1 (100%). A value of 1 would mean that “n” ppm of NH₃ would be reported as “n” ppm of NOₓ. A value of 0.5 would mean “n” ppm of NH₃ would be reported as “0.5×n” ppm of NOₓ. Sensor NOₓ2 is equipped with a NH₃ pre-filter, effectively reducing cross-sensitivity to NH₃ to substantially zero.

Turning now to the enclosed drawing, a system with the following properties is used as an example:
Exhaust Gas: (50 ppm NOₓ & 20 ppm NH₃) with value c₁=0.32
Direct differential measurement between Pt.1 and Pt.2 reads NOₓ=50 ppm
Direct differential measurement between Pt.3 and Pt.4 reads NH₃=20 ppm
Two NOₓ sensors (NOₓ1, NOₓ2) output current (Iₐ, I_{b}) that is translated to voltages (Vₐ, V_{b}) that are used as inputs into the system above.
R₁, R₂, R₃, R₄ chosen so that: R₁=R₂=R₃=R₄
R_{f} & Rᵢ chosen so that:

$$\frac{1}{{c}_{1}} = 1 + \frac{{R}_{f}}{{R}_{i}}$$

Rₐ & R_{b} chosen so that:

$$\frac{{R}_{b}}{{R}_{a} + {R}_{b}} = {c}_{1}$$

Where sensor NOₓ1 having a c₁ value of 0.32 and a sensor NOₓ2 pre-filtered for NH₃, then NH₃ is found:

NH₃=(NOₓ1−NOₓ2)/c₁

NH₃=(56.4 —50)/0.32

NH₃=6.4/0.32

NH₃=20 ppm

### To get NOₓ:

NOₓ=NOₓ1−c₁ (NH₃)

NOₓ=56.4−0.32 (20)

NOₓ=50 ppm

1. A method for simultaneously measuring two properties associated with an exhaust gas mixture, said method comprising: at least a first sensor wherein said first sensor exhibits a known cross-sensitivity to a first property and a second property in said exhaust gas mixture; a second sensor sensitive only to said first property; directly reading, between said first sensor and said second sensor, a differential value indicative of the variant concentrations of said first property and said second property, considerate of the cross-sensitivity of said first sensor to said second property.

2. The method of claim 1 wherein said first property comprises NOₓ and said second property comprises NH₃.

3. The method of claim 1, wherein said first sensor is a NOₓ sensor with a known cross-sensitivity to NH₃; said second sensor is a NOₓ sensor having no substantial cross-sensitivity to NH₃.

4. The method of claim 1, wherein said first sensor is a NOₓ sensor with a known cross-sensitivity to NH₃; said second sensor is a NOₓ sensor having no substantial cross-sensitivity to NH₃; said second sensor integrating a NH₃ pre-filter.

5. A method for simultaneously measuring NOₓ and NH₃ in an exhaust gas mixture, said method comprising: the placement of at least a first NOₓ sensor with a known cross-sensitivity to NH₃ in said exhaust gas mixture; a second NOₓ sensor with no substantial cross-sensitivity to NH₃; directly reading, between two of said sensors, a differential value proportional to the concentrations of said NOₓ and said NH₃ in said exhaust gas mixture.

6. A method for simultaneously measuring NOₓ and NH₃ in an exhaust gas mixture, said method comprising: the placement of at least a first NOₓ sensor with a known cross-sensitivity to NH₃ in said exhaust gas mixture; a second NOₓ sensor with no substantial cross-sensitivity to NH₃; directly reading, between two of said sensors, a differential value proportional to the concentrations of said NOₓ and said NH₃ in said exhaust gas mixture considerate of the cross-sensitivity of said first sensor to NH₃.

7. The method of claim 3 further comprising: configuring at least one of said NOₓ sensors to comprise a zirconia-based multilayer sensing element.

8. The method of claim 1 wherein at least one of said sensors among said plurality of sensors comprises an electrically-based sensor.
